(19) United States
(12) Patent Application Publication
(10) Pub. No.: US 2008/0009774 A1
(43) Pub. Date: Jan. 10, 2008

(54) Methods of diminishing permanent tissue markings and related apparatus

(76) Inventors: Christopher C. Capelli, Houston, TX (US); Michael B. Chancellor, Pittsburgh, PA (US)

Correspondence Address: ECKERT SEAMANS CHERIN & MELLOTT, 600 GRANT STREET, 44TH FLOOR, PITTSBURGH, PA, 15219, US

(21) Appl. No.: 11/811,507

(22) Filed: Jun. 11, 2007

Related U.S. Application Data
(60) Provisional application No. 60/813,850, filed on Jun. 15, 2006.

(51) Int. Cl. A61N 7/02

(52) U.S. Cl. 601/3

(57) ABSTRACT

A method of diminishing permanent tissue markings on a person caused by particles in the dermis includes applying ultrasound radiation to the tissue to generate cavitation bubbles and altering the tissue marking particles by collapse of the cavitation bubbles and transferring energy to the particles. The ultrasound radiation in one embodiment may have a frequency of about 15 KHz to 2 MHz and may be pulsed. The process may be repeated at the same location or other locations while resisting undesired, excessive bleeding of the dermis. The permanent tissue markings may be tattoos. The method may be used in conjunction with other methods of removing the permanent tissue markings, which may include laser, chemical agents, and biological agents. Related apparatus is enclosed.

### RELATED APPLICATION

The present application claims the benefit of U.S. Provisional Patent Application Ser. No. 60/813,850, which was filed on Jun. 15, 2006, which application is expressly incorporated herein.

### BACKGROUND OF THE INVENTION

1. Field of the Invention

A method of and related apparatus for diminishing permanent tissue markings, such as tattoos, through the use of therapeutic ultrasound. The ultrasound radiation is capable of altering pigment particles used to make tissue markings. The ultrasound radiation may have a frequency in the range of about 15 KHz to 2 MHz and is applied at an intensity and for a period of time effective to generate cavitation bubbles, wherein said cavitation bubbles collapse and transfer their energy into said tissue marking particles resulting in the alteration of said particles.

2. Description of the Prior Art

Humans have been applying tattoos to the skin for over 8000 years. The inks and dyes used were historically derived from substances found in nature and comprise a heterogeneous suspension of pigmented particles and other impurities. A well-known example is India ink, a suspension of carbon particles in a liquid.

Tattoos are produced by applying tattoo ink into the dermis, where the ink remains permanently. This technique introduces the pigment suspension through the skin by an alternating pressure-suction action caused by the elasticity of the skin in combination with the up-and-down movement of the needles. Water and other carriers for the pigment introduced into the skin diffuse through the tissues and are absorbed. The insoluble pigment particles are deposited in the dermis where initially placed, for the most part. After tattooing into the skin, the pigment particles and agglomerates are exclusively found in the cell's cytoplasm, lying in the membrane-bound structures, identified as secondary lysosomes. This finding is due to active phagocytosis into dermal cells (macrophages, fibroblasts). The resulting pigment agglomerates range up to a few micrometers in diameter. Baumler, W., et al. Q-switched laser and tattoo pigments: First results of the chemical and photophysical analysis of 41 compounds. Lasers in Surgery and Medicine 26:1321(2000). Once the skin has healed, most pigment particles remain in the interstitial space of the tissue. Inks used for tattooing resist elimination by virtue of their inertness and the relatively large size of the insoluble pigment particles. A tattoo produced in this manner will partially fade over time and will generally remain present throughout the life of the tattooed person.

Studies have been performed analyzing the make-up of tattoo inks. In a study by Tinko A. L., et al. In Vitro Quantitative Chemical Analysis of Tattoo Pigments. Arch Derinato/Vol 137, February 2001, 143-147, samples of 30 tattoo inks were examined using “standardless” energy dispersive spectrometry. Of the 30 tattoo inks studied, the most commonly identified elements were aluminum (87% of the pigments), oxygen (73% of the pigments), titanium (67% of the pigments), and carbon (67% of the pigments). The relative contributions of elements to the tattoo ink compositions were highly variable between different compounds.” See generally U.S. Pat. Nos. 5,071,422; 5,618,275; 5,827,204; 6,096,029; and 6,350,245.

In a study by Baumler, W., et al. Q-switched laser and tattoo pigments: First results of the chemical and photophysical analysis of 41 compounds. Lasers in Surgery and Medicine 26:1321(2000), it was found that the diameters of the pigments vary from about 20 nm to 900 nm. Transmission electron microscopy pictures of the pigments showed a variety of shapes such as needles, platelets, cubes bars, and a number of irregular shapes. Besides primary particles, aggregates composed of primary particles grown together at their surfaces and agglomerates (groups of single crystals joined together at their edges) are present in the same picture.”

In all types of conventional tattooing (decorative, cosmetic, and reconstructive), once the pigment or dye has been administered into the dermis to form a tattoo, the pigment or dye generally remains permanently in place. However, many people have a change of heart after being tattooed. For example, a person may desire to remove or change the design of a decorative tattoo. Alternatively, an individual with cosmetic tattooing, such as eyeliners, eyebrows, or lip coloring, may wish to change the color or area tattooed as fashion changes.

Unfortunately, there is currently no simple and successful way to remove tattoos. One approach that has been disclosed in the prior art is the use of tattoo inks that are removable on demand. These inks consist of microparticles that are constructed with specific electromagnetic absorption and/or structural properties that facilitate changing and/or removal by applying specific energy (such as electromagnetic radiation from a laser or flash-lamp). (See U.S. Patent Publication No. 2003/0167964.) In other embodiments, these pigment vehicles composed of the pigment are such that they are susceptible to a specific externally applied energy source, such as thermal, sonic (ultrasound), light (e.g., laser light, infrared light, or ultraviolet light), electric, magnetic, chemical, enzymatic, mechanical, or any other type of energy or combination of energies.

The problem with this approach is that it requires tattoos, or skin markings, to use these new types of ink. Unfortunately, tattoos using the currently available inks still will have difficulties in removal and discussed hereinbefore. Additionally, these new proposed inks are not widely used, and therefore, do not help those individuals who are seeking removal of tattoos that are made using traditional inks.

Currently, approaches to the removal of the pigment-containing skin include salabrasion, cryosurgery, surgical excision, and CO2-laser. However, these methods require invasive procedures associated with potential complications, such as infections, and usually results in conspicuous scarring.

More recently, the use of Q-switched lasers has gained wide acceptance. Ross, E. V., et al. Comparison of Responses of Tattoos to Picosecond and Nanosecond Q-Switched Neodymium:YAG Lasers. Arch Dermatol 134: 167-171 (1998). By restricting pulse duration, ink particles reach very high temperatures with relative sparing of adjacent normal skin. This significantly decreases the scarring that often results after nonselective tattoo removal methods, such as dermabrasion or treatment with carbon dioxide laser.

The mechanisms for tattoo removal by -Q-switch laser radiation are poorly understood. It is thought that Q-switch laser allow for more specific removal of tattoos by the mechanisms of selective photothermolysis and thermokinetic selectivity. Solis R. R., et al. Experimental nonsurgical tattoo removal in a guinea pig model with topical imiquimod and tretinoin. Dermatol Surg 2002;28:83-877.

While the Q-switch laser is helpful in the removal of tattoos, it is far from perfect. Some tattoos are clinically resistant to all laser therapies despite the predicted high particle temperatures achieved through selective photothermolysis. Reasons cited for failure of some tattoos to clear include the absorption spectrum of the pigment, the depth of pigment, and the structural properties of the ink.

Common adverse effects following laser tattoo treatment with the Q-switched ruby laser include textural change, scarring, and pigmentary alteration. Transient hypopigmentation and textural changes have been reported in up to 50 and 12%, respectively, of patients treated with the Q-switched alexandrite laser. Hyperpigmentation and textural changes are infrequent adverse effects of the Q-switched Nd:YAG laser and the incidence of hypopigmentary changes are much lower than with the ruby laser. The development of localized and generalized allergic reactions is an unusual complication following tattoo removal with the Q-switched ruby and Nd:YAG lasers. Kuperman-Beade M., et al. Laser removal of tattoos. Am J Clin Dermatol. 2001;2 (1):21-5.

Additionally, laser treatment is painful. Treatment with the Q-switch laser is painful. Local injection with lidocaine or topical anesthesia cream typically is used prior to laser treatment. Kuperman-Beade M., et al. Laser removal of tattoos. Am J Clin Dermatol. 2001;2 (1):21-5.

Finally, laser removal requires multiple treatment sessions (usually five to twenty) with expensive equipment for maximal elimination. Typically, as many wavelengths are needed to treat multicolored tattoos, not one laser system can be used alone to remove all the available inks and combination of inks. Kuperman-Beade M., et al. Laser removal of tattoos. Am J Clin Dermatol. 2001;2 (1):21-5. Even with multiple treatments, laser therapy is usually limited to eliminating only from 50-70% of the tattoo pigment, resulting in a residual smudge. As a result, the overall cost of laser removal is generally prohibitively expensive.

Ultrasound is best known for its imaging capability in diagnostic medicine. However there have been considerable efforts recently to develop therapeutic uses for ultrasound. A review by Ka-yun Ng and Yange Liu detailed the applications of ultrasound to enhance the delivery and effect of three distinctive therapeutic drug classes: chemotherapeutic, thrombolytic and gene-based drugs. Ng K. Y., et al. Therapeutic ultrasound: its application in drug delivery, Med Res Rev. March 2002;22(2):204-23.

Currently, substantial research is being performed on the use therapeutic ultrasound for drug delivery. For example, U.S. Pat. No. 5,618,275 discloses an invention that relates to a method of facilitating the penetration of a therapeutic agent through a person's skin comprising, applying relatively low frequency ultrasonic pressure waves in a range of about 15,000 to about 25,000 Hz to the skin of sufficiently high intensity to cause cavitation in the skin. The effect of the low frequency ultrasonic pressure waves is to increase the permeability of the skin which allows the penetration of therapeutic agents for a limited time period.

U.S. Pat. No. 6,487,447 discloses an apparatus for performing in-vivo sonoporation of a skin area and transdermal and/or intradermal delivery of a drug solution. The apparatus includes a container having an end covered with a porous membrane and containing the drug solution and an ultrasound horn having a tip submerged in the drug solution. The ultrasound horn applies ultrasound radiation to the drug solution. The ultrasound radiation has a frequency in the range of about 15 KHz to 2 MHz and is applied at an intensity, for a period of time and at a distance from said skin area effective to generate cavitation bubbles. The cavitation bubbles collapse and transfer their energy into the skin area thus causing the formation of pores in the skin area.

There is a real and substantial need for a non-surgical method for removal of tissue markings that is not dependent on the transmission and absorption of laser light.

### SUMMARY OF THE INVENTION

A method and apparatus are provided for the diminishment of permanent tissue markings, such as tattoos, through the use of therapeutic ultrasound. The ultrasound radiation is capable of efficient alteration of pigment particles used to make tissue markings. This invention provides an apparatus that can generate ultrasonic radiation at the lower ultrasonic frequencies while supplying sufficient ultrasonic power so that the desired particle alteration through cavitation is obtained without significant heating of the tissues of the patient.

The ultrasound radiation that is used in this invention is capable of altering pigment particles used to make tissue markings. The ultrasound radiation preferably has a frequency in the range of 15 KHz and 2 MHz and is applied at an intensity and for a period of time effective to generate cavitation bubbles. The cavitation bubbles collapse and transfer their energy into said tissue marking particles resulting in the alteration of said particles. In one embodiment of this invention, the ultrasound radiation is applied in a pulsed fashion.

It is an object of the present invention to provide a method and associated apparatus for diminishing permanent tissue markings, such as tattoos, through the use of therapeutic ultrasound.

It is another object of the present invention to provide such a system which employs ultrasound radiation to alter the pigment particles employed to establish the tissue markings.

It is a further object of the present invention to provide such a system which will safely, economically, and efficiently remove at least significant portions of the tissue markings.

It is a further object of the present invention to provide such a system which will be effective without requiring a large number of repeat treatments.

These and other objects of the invention will be more fully understood from the following detailed description of the invention when referenced to the illustrations appended hereto.

### BRIEF DESCRIPTION OF THE DRAWINGS

FIG. 1 is a schematic illustration of a form of apparatus employable in the present invention.

FIG. 2 is a plot representative of ultrasound wave cycles useful in the present invention.

### DESCRIPTION OF THE PREFERRED EMBODIMENTS

This invention relates to a method and an ultrasonic apparatus for the diminishment of permanent tissue markings, such as tattoos, through the use of therapeutic ultrasound. The ultrasound radiation that is used in this invention is capable of altering pigment particles used to make tissue markings. The ultrasound radiation has a frequency in the range of 15 KHz and 2 MHz and is applied at an intensity and for a period of time effective to generate cavitation bubbles, wherein said cavitation bubbles collapse and transfer their energy into said tissue marking particles resulting in the alteration of said particles. As used herein, “therapeutic ultrasound” encompasses all non-diagnostic ultrasound modalities.

Furthermore, as used herein, altering and alteration of particles refers to the disruption, fragmentation, erosion, or other modification of particles so as to become less visible when used for tissue markings.

Cavitation is the formation and activity of bubbles or cavities in a liquid. More specifically, cavitation is the formation and collapse of microbubbles in a fluid due to pressure in the fluid reaching certain critical levels. Grandia W., et al. Medical Noninvasive Operations using focused modulated high power ultrasound. U.S. Pat. No. 5,827,204, Oct. 27, 1998.

Ultrasonic cavitation is a sonically induced formation and activity of bubbles or cavities in a liquid. Two types of cavitation can be distinguished, and the biological effect depends broadly on which type occurs. The first type is stable or non-inertial cavitation. This type of cavitation refers to the rhythmic oscillation in size of a bubble in an ultrasonic field. A number of effects may be related to stable cavitation, including translational motion of free bubbles, bubble surface distortions, growth by rectified diffusion or by coalescence, heat generation, and radiation forces exerted on neighboring cells and small-scale acoustic streaming around the bubbles. Moreover, cell membranes may become transiently permeable to larger molecules such as DNA. This is called sonoporation.

The second type of cavitation is inertial or collapse cavitation. This type of cavitation is the most destructive type. This term is used to refer to the expansion of nuclei during the low pressure phase and the following collapse to a small fraction of its original volume during the compression phase. Combined with the collapse, local pressures of 100 MPa as well as temperature rises well over 1000 K can occur.

Inertial cavitation can generate microscopic shock waves which propagate into the fluid surrounding the collapsing microbubble. If bubbles are bound to solid surfaces, destructive microjets of liquid may be directed towards the solid causing surface damage. The bubbles often tend to form in or migrate into cracks on the particle surfaces and sometimes lead to the breakage of particles due to rapid bubble dynamics. Wolfrum B., et al. Shock wave induced interaction of microbubbles and boundaries. Physics of Fluids, Vol. 15, No. 10 October 2003. A study by Malykh N V et al demonstrated that particles of different initial sizes (1 μm to 300 μm) and different materials experienced rapid disintegration by ultrasonic cavitation. Malyhk H. V., et al. Ultrasonic cavitational chemical technologies, XI Session of the Russian Acoustical Society, Moscow, Nov. 19-23, 2001.

In addition to a method, this invention embodies a therapeutic ultrasound apparatus for facilitating the diminishment of permanent tissue markings caused by particles through application of ultrasound radiation. This therapeutic ultrasound apparatus comprises, 1) a housing, said housing supporting a piezoelectric transducer including at least one-active element for contracting and expanding volumetrically when energized in response to a changing electrical field and generating vibrations of ultrasonic radiation; 2) an ultrasonic power supply to generate ultrasonic frequency electric signals; and 3) a means coupling said power supply to said piezoelectric transducer. The therapeutic ultrasound apparatus provides ultrasound radiation that has a frequency in the range of about 15 KHz to 2 MHz and is applied at an intensity and for a period of time effective to generate cavitation bubbles, wherein said cavitation bubbles collapse and transfer their energy into said tissue marking particles resulting in the alteration of said particles.

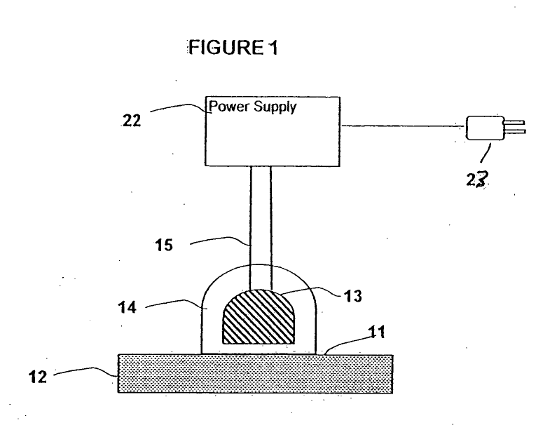

The therapeutic ultrasonic apparatus, in accordance with an embodiment of the present invention is shown in FIG. 1. This apparatus comprises a piezoelectric transducer 13 and connecting wiring 15 that connects the transducer 13 to the external ultrasonic power supply 22. The external power supply 22 draws its power either from a standard household current through a connector 23 or is operated from a battery within the power supply 22. The piezoelectric transducer 13 is encapsulated in a housing 14 made of a rigid material such as plastic shown in position on top of the outer surface II of the skin 12.

To provide ultrasonic radiation useful for the destruction of tissue marking particles, this invention provides an ultrasound signal with a frequency in the range of 15 KHz to 2 MHz. The preferred ultrasound intensity for the destruction of tissue marking particles may be in the range of 5 W/cm² and 100 W/cm².

The majority of ultrasonic power at the higher frequencies is absorbed in the tissue in the form of heat, creating unsatisfactory thermal injury to tissues if the power density is large enough. Bock R T, Ultrasonic method and apparatus for cosmetic and dermatological applications. U.S. Pat. No. 5,618,275. To reduce the effects of thermal damage to tissues from the absorption of high frequency ultrasound, the preferred frequency for the ultrasound radiation is in the range of about 15 KHz to 100 KHz.

Most preferably, the therapeutic ultrasound of this invention provides a frequency signal at a level slightly below that is required for causing cavitation, i.e. below the cavitation threshold, in the targeted tissue. When the targeted tissue has tissue marking particles present, these particles act as nucleation points for the ultrasonic radiation resulting in cavitation that acts to alter said particles. Upon the complete dissolution of the particles, cavitation ceases. If the targeted tissue being treated with the therapeutic ultrasound does not have tissue marking particles, there is only minimal cavitation generated.

The ultrasonic radiation for the alteration of tissue marking particles can be applied either in a continuous wave ultrasonic modality, or in the form ultrasonic wave cycle that consists of periodic pulses of ultrasound. Preferably, the ultrasonic radiation is applied in the form of ultrasonic wave cycle to reduce heat buildup in the tissue.

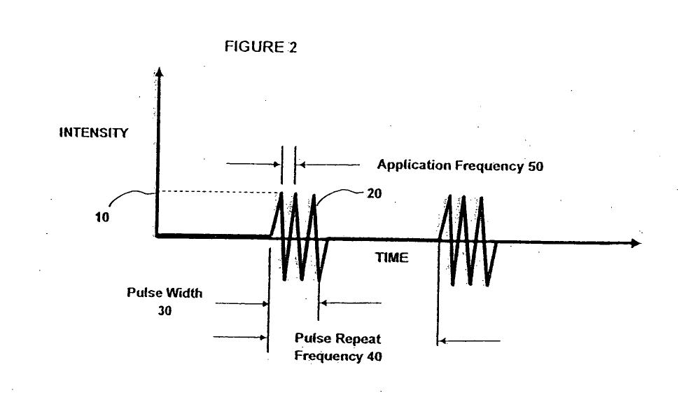

As a means of illustration, an ultrasonic wave cycle that could be useful for this invention is illustrated in FIG. 2. The ultrasound wave cycle has pulses 20 having a frequency of 20 KHz and intensity 10 of 20 W/cm². The pulse width 30 is 0.5 seconds, the time interval 40 between the end of one pulse and the beginning of the next is 9.5 seconds and the period of the ultrasound wave is 10 seconds. In one example, the skin is exposed to ultrasound for 20 minutes with a 5% duty cycle, i.e., 120 pulses with each pulse providing ultrasound energy for 0.5 seconds, resulting in a total of 1 minute of continuous ultrasound exposure. Weimann, L., et al. Method and apparatus for in-vivo transdermal and/or intradermal delivery of drugs by sonoporation, U.S. Pat. No. 6,487,447, Nov. 26, 2004.

The method of diminishing tissue marking caused by pigments that are placed in dermis tissue would involve the using a therapeutic ultrasonic apparatus or system as described above, i.e. a piezoelectric transducer, connecting wiring, and the external ultrasonic power supply, that has a transducer to produce ultrasonic radiation continuously or preferably in the form of an ultrasonic wave cycle. This ultrasonic radiation may be unfocused, or preferably, focused within the dermal layer that contains the pigment. The ultrasonic radiation is transmitted into the patient's skin. When said ultrasonic radiation comes in contact with the intradermal particles, these particles act as nucleation points resulting in cavitation at the particle surface leading to the alteration of said particles.

The present invention involves the use of ultrasound to alter intradermal microscopic particles, including pigment particles used for tattoos, through cavitation. The microscopic particles are effectively altered using cavitation. The example in this invention demonstrates that microscopic particles that make up tattoo inks can be made smaller after being treated with ultrasonic radiation. Theoretically, the pigment particles are reduced to small particles, which are then made less visible and/or absorbed by cells and eliminated.

In diminishing skin markings, the patient would have the ultrasonic transducer from the therapeutic ultrasound apparatus placed directly on the tissue site having the tissue markings. In the case of where the tissue marking is a tattoo, the ultrasonic transducer is placed directly on the skin containing the tattoo.

Likewise, the ultrasonic transducer can be placed on a coupling media such as a water balloon or hydrogel that is then placed on the tissue site. Preferably, the therapeutic ultrasound has a transducer and/or coupler that are designed to focus the ultrasonic radiation in the dermis of the patient. Preferably, the focus of the ultrasonic radiation should provide coverage of an area greater than 5 mm in diameter and most preferably greater than 10 mm in diameter. The desired ultrasonic wave cycle is one that provides minimal cavitation in tissue but maximum cavitation at the site of particles in tissues. Most preferred is an ultrasonic wave cycle that provides minimal heating of tissue while having the ability to form cavitation in the presence of particles. To achieve these goals, the preferred ultrasonic wave cycle should be in the form of pulses to minimize tissue heating with a frequency of less then 100 KHz.

To cause particle alteration, the ultrasound radiation typically needs to be delivered to a specific area for a period of time long enough to affect the pigment particles.

Additionally, tattoos typically cover large areas of dermis. As the therapeutic ultrasound has a focus area that is relatively small in comparison to the typical tattoo, the therapeutic ultrasound will have to be moved to different areas in order to remove the tattoo. As a result, it is preferred that the therapeutic ultrasound be focused at different areas of the tattoo periodically. This has the added benefit of minimizing potential discomfort caused by cavitation bubbles by providing time for said bubbles to be reabsorbed in the area of the tissue that was just undergone a period of ultrasonic radiation. The therapeutic ultrasound would provide a ultrasonic radiation to one area and then be moved to a second area, third area, etc. After providing ultrasonic radiation to the different areas, the ultrasound would begin the series again starting with the first area. This process can be done automatically through the programming of the therapeutic ultrasound or having an ultrasonic transducer that can be directed electronically or mechanically. In this way, efficient use of the therapeutic ultrasound apparatus is obtained while providing extended interval time between pulses at a specific tattoo area—that is being treated. It should be noted that for small tattoos, a single area can be treated. In this case, care must be taken to provide an adequate time interval between pulses so as to minimize the formation of bubbles.

The use of therapeutic ultrasound for the removal of skin markings has many potential major advantages over the use of lasers. In general laser treatment for tattoo removal is painful. The use of therapeutic ultrasound may provide removal of tattoos with little if any pain. This is particularly true when therapeutic ultrasound is used at a lower frequency and is pulsed.

The use of laser light directed at tissue has been found to cause damage to or destruction of the surrounding tissues. Therapeutic ultrasound for tattoo removal will generate little heat and therefore result in little damage or destruction of the surrounding tissues.

Finally, laser removal requires multiple treatment sessions, usually five to twenty, with expensive equipment for maximal elimination. Typically, as many wavelengths are needed to treat multicolored-tattoos, more than one laser system is needed to remove all the available inks and combination of inks. Weimann L., et al. Method and apparatus for in-vivo transdermal and/or intradermal delivery of drugs by sonoporation. U.S. Pat. No. 6,487,447. Even with multiple treatments, laser therapy is usually limited to eliminating only from 50-70% of the tattoo pigment, resulting in a residual smudge. As a result, the overall cost of laser removal is generally prohibitively expensive. Therapeutic ultrasound on the other hand can be applied to a relatively large area in an efficient manner thereby reducing the number of treatment sessions. Furthermore, as therapeutic ultrasound is not dependent on the absorption of light, separate systems are not required to remove all the available inks and combination of inks.

This invention provides a method for the diminishment of permanent tissue markings, such as tattoos, through the use of therapeutic ultrasound. There may be situations wherein a combination of approaches is used to remove the permanent tissue markings. For example, this invention contemplates the use of method of diminishment of permanent tissue marking caused by particles through the use of therapeutic ultrasound in combination with laser such as a Q-switch laser.

Furthermore, this invention also contemplates the use of a method of diminishing permanent tissue marking caused by particles through the use of therapeutic ultrasound in combination with a chemical or biological agents that can aid in the removal of the tissue marking. For example, the use of a chelator (e.g., EDTA) or an immune modulator (e.g., Solis R R, Dayna D G, Colome-Grimmer M O. Wagner, Snyder M. RF: Experimental nonsurgical tattoo removal in a guinea pig model with topical imiquimod and tretinoin, Dermatol Surg 2002;28:83-877) is contemplated These chemical or biological agents could be delivered transdermally or systemically. The agents can be provided prior to, during or following the treatment with the ultrasonic radiation.

While particular embodiments of the present invention have been described herein for purposes of illustration, it will be evident to those skilled in the art that numerous variations of the details can be made without departing from the invention as set forth in the appended claims.

What is claimed is:

1. A method of diminishing permanent tissue markings on a person caused by particles in the dermis comprising:
applying ultrasound radiation to said tissue to generate cavitation bubbles, and altering said particles by collapse of said cavitation bubbles and transfer of energy to said tissue marking particles.

2. The method of claim 1 including:
employing ultrasonic energy in the range of about 15 KHz to 2 MHz.

3. The method of claim 1 including:
employing pulsed ultrasonic energy as said ultrasound radiation.

4. The method of claim 1 including:
repeating said process at the same tissue marking location.

5. The method of claim 4 including:
prior to said repeating of said process at the same location performing said process at another location on said person.

6. The method of claim 4 including:
repeating said process at another location on said person to resist undesired heating of said person in the first location.

7. The method of claim 1 including:
employing said method to diminish a tattoo.

8. The method of claim 1 including:
employing said method in conjunction with other means to diminish said permanent tissue markings.

9. The method of claim 8 including:
employing a laser as said other means.

10. The method of claim 8 including:
employing at least one agent selecting from the group consisting of chemical agents and biological agents as said other means.

11. Apparatus for diminishing permanent tissue markings on a person caused by particles in the dermis comprising:
a piezoelectric transducer structured to generate vibrations of ultrasonic energy in the range of about 15 KHz to 2 MHz,
an ultrasonic power supply operatively associated with said piezoelectric transducer for generating ultrasonic frequency electrical signals, and
said piezoelectric transducer structured to generate cavitation bubbles which collapse and transfer energy into the said tissue marking particles to alter said particles.

12. The apparatus of claim 1 I including:
said apparatus being structured to create ultrasound radiation at an intensity and for a period of time effective to generate said cavitation bubbles.

13. The apparatus of claim 12 including:
said apparatus structured to provide pulsed ultrasound radiation.

14. The apparatus of claim 12 including:
said apparatus structured to diminish a permanent tissue marking which is a tattoo.
